(19) United States
(12) Patent Application Publication
(10) Pub. No.: US 2024/0156862 A1
(43) Pub. Date: May 16, 2024

(54) TREATMENT OF CHRONIC OBSTRUCTIVE PULMONARY DISEASE WITH MYELOID DERIVED SUPPRESSOR CELLS

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US)

(73) Assignee: Therapeutic Solutions International, Inc., Oceanside, CA (US)

(21) Appl. No.: 18/508,623

(22) Filed: Nov. 14, 2023

Related U.S. Application Data
(60) Provisional application No. 63/425,259, filed on Nov. 14, 2022.

(51) Int. Cl. A61K 35/15; A61P 11/00; C07K 16/24; C12N 5/078

(52) U.S. Cl. CPC A61K 35/15; A61P 11/00; C07K 16/244; C12N 5/0634; C12N 2501/22; C12N 2501/2303

(57) ABSTRACT

Disclosed are compositions of matter, protocols, and treatment means for prevention and/or reversing Chronic Obstructive Pulmonary Disease (COPD) using myeloid derived suppressor cells as a monotherapy or adjuvant therapy. In one embodiment umbilical cord low density myeloid cells are expanded using interleukin-3 and GM-CSF and administered in an allogeneic manner to a mammal suffering from COPD. In some embodiments combinations of myeloid derived suppressor cells and mesenchymal stem cells are disclosed.

### CROSS-REFERENCE TO RELATED APPLICATIONS

This application claims priority to U.S. Provisional Application No. 63/425,259, filed Nov. 14, 2022, and titled: “Treatment of Chronic Obstructive Pulmonary Disease with Myeloid Derived Suppressor Cells”, which is hereby incorporated by reference in its entirety.

### BACKGROUND OF THE INVENTION

Chronic obstructive pulmonary disease (COPD) is a common, preventable and untreatable chronic lung disease which affects men and women worldwide. Abnormalities in the small airways of the lungs lead to limitation of airflow in and out of the lungs. Several processes cause the airways to become narrow. There may be destruction of parts of the lung, mucus blocking the airways, and inflammation and swelling of the airway lining. COPD is sometimes called emphysema or chronic bronchitis. Emphysema usually refers to destruction of the tiny air sacs at the end of the airways in the lungs. Chronic bronchitis refers to a chronic cough with the production of phlegm resulting from inflammation in the airways.

New methods and compounds are needed to prevent or reverse COPD. The invention provides the use of immunomodulatory and regenerative cells for this purpose.

### FIELD OF THE INVENTION

The invention herein relates to the use of immunomodulatory cells to treat and/or prevent chronic obstructive pulmonary disease (COPD).

### SUMMARY

Preferred embodiments include methods of treating chronic obstructive pulmonary disease (COPD) comprising the steps of: a) obtaining a patient suffering from COPD; b) assessing inflammatory markers in said patient; c) administering to said patient a therapeutic dose of myeloid derived suppressor cells; and d) assessing patient pathology and inflammatory markers and adjusting dose and/or frequency of myeloid derived suppressor cells being administered.

Preferred methods include embodiments wherein said inflammatory markers are cytokines capable of inducing activation of NF-kappa B.

Preferred methods include embodiments wherein said inflammatory markers are selected from the group consisting of: a) interleukin-1; b) TNF-alpha; c) interleukin-6; d) interleukin-17; e) interleukin-18; 0 interleukin-33, g) HMGB-1, and h) interleukin-10.

Preferred methods include embodiments wherein said myeloid derived suppressor cell expresses a marker selected from the group consisting of: CD133, CD33, and CD34.

Preferred methods include embodiments wherein the method further comprises administering to the patient an antibody to said inflammatory marker.

Preferred methods include embodiments wherein the inflammatory marker is interleukin-10.

Preferred methods include embodiments wherein said myeloid derived suppressor cell is generated by culture of myeloid cells in the presence of interleukin-3.

Preferred methods include embodiments wherein said myeloid derived suppressor cell is generated by culture of myeloid cells in the presence of interleukin-10.

Preferred methods include embodiments wherein said myeloid derived suppressor cell is generated by culture of myeloid cells in the presence of GM-CSF.

Preferred methods include embodiments wherein said myeloid derived suppressor cell is generated by culture of myeloid cells in the presence of interleukin-3 and GM-CSF.

Preferred methods include embodiments wherein said myeloid derived suppressor cell is generated by culture of myeloid cells in the presence of interleukin-3 and GM-CSF and interleukin-10.

Preferred methods include embodiments wherein said myeloid derived suppressor cell is generated by culture of myeloid cells under hypoxic conditions.

Preferred methods include embodiments wherein said myeloid derived suppressor cell is generated by culture of myeloid cells in the presence of TGF-beta.

Preferred methods include embodiments wherein said myeloid derived suppressor cell is generated by culture of myeloid cells in the presence of prostaglandin E2.

Preferred methods include embodiments wherein said myeloid derived suppressor cell is obtained from umbilical cord tissue.

Preferred methods include embodiments wherein said myeloid derived suppressor cell is obtained from placental tissue.

Preferred methods include embodiments wherein said myeloid derived suppressor cell is generated from umbilical cord blood.

Preferred methods include embodiments wherein said myeloid derived suppressor cell is generated peripheral blood.

Preferred methods include embodiments wherein said myeloid derived suppressor cell is generated from mobilized peripheral blood.

Preferred methods include embodiments wherein said myeloid derived suppressor cell is activated by pretreatment with a toll like receptor agonist.

### BRIEF DESCRIPTION OF THE DRAWINGS

FIG. 1 is a bar graph showing the effects of myeloid suppressor cells and/or IL-10 antibodies on inhibition of inflammation and COPD pathology.

### DETAILED DESCRIPTION OF THE INVENTION

For the purpose of the invention, we disclose that two populations of myeloid derived suppressor cells are capable of stimulating regeneration of injured or degenerated lung tissue. In one embodiment the invention provides myeloid derived suppressor cells for treatment of COPD.

The first type of myeloid derived suppressor cells is the monocytic myeloid-derived suppressor cells (M-MDSC) and the second type is polymorphonuclear myeloid-derived suppressor cells (PMN-MDSC). About 20-30% of MDSC consists of monocytic cells, i.e., M-MDSC, and are generally associated with high activity of Arginase-1 and iNOS.sup.10. Two different phenotypes (CD11b.sup.+CD14.sup.−CD15.sup.− and CD33.sup.+ or CD11b.sup.+CD14.sup.+CD33.sup.+ and HLA-DR.sup.lo) are used to characterize these M-MDSC cells depending on the type of cancer. The second population, i.e., PMN-MDSC, are comprised of granulocytic cells and are usually associated with high level of ROS production.sup.36. PMN-MDSC represent the major population of MDSC (about 60-80%) and represent the most abundant population of MDSC in most types of cancer. PMN-MDSC are phenotypically and morphologically similar to neutrophils (PMN) and share the CD11b+CD14-CD15+/CD66b+ phenotype. The may also be characterized as CD33.sup.+. PMN-MDSC are important regulators of immune responses in cancer and have been directly implicated in promotion of tumor progression. However, the heterogeneity of these cells and lack of distinct markers hampers the progress in understanding of the biology and clinical significance of these cells. One of the major obstacles in the identification of PMN-MDSC is that they share the same phenotype with normal polymorphonuclear cells (PMN).

The administration of myeloid derived suppressor cells may be performed by using the cells themselves or by pre-activating them. In certain embodiments of the invention, small molecules of the invention are used to sustain and enhance the immune suppressive functions of MDSCs by preventing the MDSCs to undergo maturation and terminal differentiation. Through this process the growth factor producing properties of the myeloid suppressor cells are retained and/or enhanced.

For the purpose of the invention, we describe the immature stage of MDSCs as being characterized by low cell surface expression of MHC class II, co-stimulatory molecules, e.g., CD80, CD86, CD40, low CD11c and F4/80. Immature MDSCs arc further characterized by a large nucleus to cytoplasm ratio and an immunosuppressive activity. In some cases enhancement of growth factor properties is produced by treatment of the cells by histone deacetylase inhibitors such as decitabine. In some embodiments of the invention, MDSCs are autologously-derived cells. For example, MDSCs may be isolated from normal adult bone marrow or from sites of normal hematopoiesis, such as the spleen. Obviously, splenic sources of MDSC are difficult in clinical situations. MDSCs are scant in the periphery and are present in a low number in the bone marrow of healthy individuals. However, they are accumulated in the periphery when intense hematopoiesis occurs. Upon distress due to graft-versus-host disease (GVHD), cyclophosphamide injection, or g-irradiation, for example, MDSCs may be found in the adult spleen. Thus, in certain embodiments, MDSCs may be isolated from the adult spleen. MDSCs may also be isolated from the bone marrow and spleens of tumor-bearing or newborn mice. In a preferred embodiment, MDSCs are isolated in vivo by mobilizing MDSCs from hematopoietic stem cells (HSCs) or bone marrow suing stem cell mobilizers such as G-CSF Any suitable stem cell mobilizer or combination of mobilizers is contemplated for use in the present invention. MDSCs may be induced endogenously and/or be collected from the blood e.g., by apheresis, following treatment of a subject or patient with the stem cell mobilizer(s). In certain embodiments, MDSCs can be derived, for example, in vitro from a patient's HSCs, from MHC matching ES cells, induced pluripotent stem (iPS) cells Specifically, isolated hematopoietic stem cells (HSCs) can be stimulated to differentiate into Gr-1+/CD11b+, Gr-1+/CD11b.+/CD115+, Gr-1+/CD11b+/F4/80+, or Gr-1+/CD11b+/CD115+/F4/80+ MDSCs by culturing in the presence of stem-cell factor (SCF) or SCF with tumor factors, which can increase the MDSC population. The culture conditions for mouse and human HSCs are described in detail in U.S. Publication No. 2008/0305079 by Chen. In further embodiments, other cytokines may be used, e.g., VEGF, GM-CSF, M-CSF, SCF, S100A9, TPO, IL-6, IL-1, PGE-2 or G-CSF to stimulate MDSC differentiation from HSCs in vitro. Any one of the cytokines may be used alone or in combination with other cytokines. In still another embodiment, tumor-conditioned media may be used with or without SCF to stimulate HSCs to differentiate into MDSCs. In other embodiments, MDSCs are allogeneic cells, such as MDSCs obtained or isolated from a donor or cell line. MDSC cell lines and exemplary methods for their generation are well known in the art and are described in the literature.

### Example

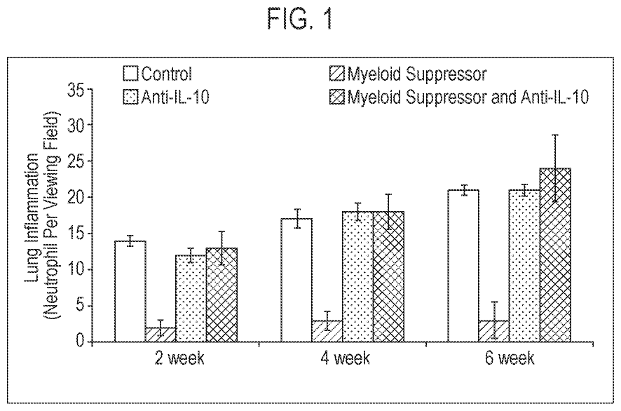

BALB/c mice were treated with 1 microgram of elastase per mouse and administered either myeloid suppressor cells generated from umbilical cord blood by treatment with IL-3 (100 ng/ml) and GM-CSF (50 ng/ml). Some mice received anti-IL-10 antibody and combination of anti-IL-10 and myeloid suppressor cells. As seen in the FIGURE below significant inhibition of inflammation and COPD pathology was observed during treatment with myeloid suppressor cells. Results are shown in FIG. 1.

1. A method of treating chronic obstructive pulmonary disease (COPD) comprising the steps of:
a) obtaining a patient suffering from COPD; b) assessing inflammatory markers in said patient;
c) administering to said patient a therapeutic dose of myeloid derived suppressor cells; and d) assessing patient pathology and inflammatory markers and adjusting dose and/or frequency of myeloid derived suppressor cells being administered.

2. The method of claim 1, wherein said inflammatory markers are cytokines capable of inducing activation of NF-kappa B.

3. The method of claim 1, wherein said inflammatory markers are selected from the group consisting of: a) interleukin-1; b) TNF-alpha; c) interleukin-6; d) interleukin-17; e) interleukin-18; 0 interleukin-33, g) HMGB-1, and h) interleukin-10.

4. The method of claim 1, wherein said myeloid derived suppressor cell expresses a marker selected from the group consisting of: CD133, CD33, and CD34.

5. The method of claim 3, wherein the method further comprises administering to the patient an antibody to said inflammatory marker.

6. The method of claim 5, wherein the inflammatory marker is interleukin-10.

7. The method of claim 6, wherein said myeloid derived suppressor cell is generated by culture of myeloid cells in the presence of interleukin-3.

8. The method of claim 1, wherein said myeloid derived suppressor cell is generated by culture of myeloid cells in the presence of interleukin-10.

9. The method of claim 7, wherein said myeloid derived suppressor cell are also generated by culture of myeloid cells in the presence of GM-CSF.

10. The method of claim 1, wherein said myeloid derived suppressor cell is generated by culture of myeloid cells in the presence of interleukin-3 and GM-CSF.

11. The method of claim 1, wherein said myeloid derived suppressor cell is generated by culture of myeloid cells in the presence of interleukin-3 and GM-CSF and interleukin-10.

12. The method of claim 1, wherein said myeloid derived suppressor cell is generated by culture of myeloid cells under hypoxic conditions.

13. The method of claim 1, wherein said myeloid derived suppressor cell is generated by culture of myeloid cells in the presence of TGF-beta.

14. The method of claim 1, wherein said myeloid derived suppressor cell is generated by culture of myeloid cells in the presence of prostaglandin E2.

15. The method of claim 1, wherein said myeloid derived suppressor cell is obtained from umbilical cord tissue.

16. The method of claim 1, wherein said myeloid derived suppressor cell is obtained from placental tissue.

17. The method of claim 1, wherein said myeloid derived suppressor cell is generated from umbilical cord blood.

18. The method of claim 1, wherein said myeloid derived suppressor cell is generated peripheral blood.

19. The method of claim 1, wherein said myeloid derived suppressor cell is generated from mobilized peripheral blood.

20. The method of claim 1, wherein said myeloid derived suppressor cell is activated by pretreatment with a toll like receptor agonist.
